(19) United States
(12) Patent Application Publication
(10) Pub. No.: US 2026/0151055 A1
(43) Pub. Date: Jun. 4, 2026

(54) Sensor insertion

(71) Applicants: KATHERINE WOLFE, DUNWOODY, GA (US); BENJAMIN PAGLIUSO, MARINA DEL REY, CA (US); BELLA NAVA, LOS ANGELES, CA (US)

(72) Inventors: KATHERINE WOLFE, DUNWOODY, GA (US); BENJAMIN PAGLIUSO, MARINA DEL REY, CA (US); BELLA NAVA, LOS ANGELES, CA (US)

(73) Assignee: PercuSense, Inc., Valencia, CA (US)

(21) Appl. No.: 18/821,957

(22) Filed: Aug. 30, 2024

Related U.S. Application Data
(60) Provisional application No. 63/535,380, filed on Aug. 30, 2023.

(51) Int. Cl. A61B 5/145; A61B 5/00

(52) U.S. Cl. CPC A61B 5/14503; A61B 5/6847; A61B 5/14532; A61B 5/14546; A61B 2560/06

(57) ABSTRACT

In one embodiment an insertion assembly to implant a sensor is disclosed. The insertion device includes an encasement with an interior defined between an encasement top and an encasement bottom. The assembly further includes a slider with a slider top and a slider bottom face. The slider top is located within the interior of encasement and the slider initially is in a first position, capable of transitioning to a second position and further capable of transitioning to a third position. Wherein in the first position the slide bottom face is coincident with the encasement bottom, in the second position the slide bottom face is fully contained in the interior, and in the third position, the slide bottom face extends beyond the encasement bottom by a gap.

### RELATED APPLICATIONS

This application claims the benefit of U.S. provisional application serial number: 63/535,380, filed on Aug. 30, 2023. The application listed above is hereby incorporated by reference in its entirety for all purposes.

### FIELD OF THE INVENTION

The present invention is generally directed to devices and methods that enable insertion of percutaneous medical devices such as, but not limited to an analyte sensor or infusion cannula into a subject. The devices and methods are intended to insert or implant a sensor percutaneously that is capable of monitoring the presence or amount of an analyte or analytes within a subject. Alternatively, the devices and methods may be used to insert or implant an infusion cannula configured to infuse fluids such as a medicant like insulin into a subject. In other embodiments the devices and methods enable the percutaneous insertion of a single device that includes both a sensor and infusion cannula.

### BACKGROUND OF THE INVENTION

In vivo monitoring of particular analytes can be critically important to short-term and long-term well being. For example, the monitoring of glucose can be particularly important for people with diabetes in order to determine insulin or glucose requirements. Additionally, the monitoring of multiple analytes associated with metabolic health, such as lactate, can provide additional insight. The need to perform continuous or near continuous monitoring of at least one analyte has resulted in the development of a variety of devices and methods. Some methods place electrochemical sensor devices designed to detect the desired analyte in blood vessels while other methods place the devices in subcutaneous or interstitial fluid.

Commercially available glucose sensors are typically placed in subcutaneous or interstitial fluid and for users, insertion of the sensor involves using an insertion device that uses a needle or sharp to pierce the skin and insert the sensor at a preferred depth below the skin. For many users, there may be a sense of anxiety associated with using the insertion device as any device piercing the skin using a needle or sharp can potentially draw blood or result in pain or discomfort. As implantable sensors become readily available to monitor general health, it may be beneficial to attempt to manage or mitigate trypanophobia that may arise because of the insertion process.

The claimed invention seeks to provide an insertion process or tool that reduces anxiety associated with inserting the sensor.

### BRIEF SUMMARY OF THE INVENTION

In one embodiment an insertion assembly to implant a sensor is disclosed. The insertion device includes an encasement with an interior defined between an encasement top and an encasement bottom. The assembly further includes a slider with a slider top and a slider bottom face. The slider top is located within the interior of encasement and the slider initially is in a first position, capable of transitioning to a second position and further capable of transitioning to a third position. Wherein in the first position the slide bottom face is coincident with the encasement bottom, in the second position the slide bottom face is fully contained in the interior, and in the third position, the slide bottom face extends beyond the encasement bottom by a gap.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1 is an exemplary pseudo-isometric view of select components of an insertion assembly, in accordance with embodiments of the present invention.

FIG. 2 is an exemplary illustration of the insertion assembly in a third position, in accordance with embodiments of the present invention.

FIG. 3A is an exemplary illustration of the multiple components associated with the insertion assembly during an assembly phase of an insertion process, in accordance with embodiments of the present invention.

FIGS. 3B and 3C are exemplary illustrations of multiple components associated with the insertion assembly during a coupling phase, in accordance with embodiments of the present invention.

FIG. 3D is an exemplary illustration of select components of the insertion assembly in a second position, in accordance with embodiments of the present invention.

FIG. 3E is an exemplary illustration of select components of the insertion assembly in a third position, in accordance with embodiments of the present invention.

### DETAILED DESCRIPTION

Presented below are embodiments of a system that is intended to insert or place an implantable sensor percutaneously into a user or subject. In many embodiments the sensor is intended to be inserted through the skin with a distal end of the sensor eventually being located within subcutaneous tissue. However, it should be understood that in other embodiments, the sensor may be placed at an alternative depth below or beneath the skin of a subject. For example, in some embodiments it may be desirable to insert the sensor into a vein or alternatively, an organ. Irrespective of the insertion depth, for many individuals, particularly those with issues with needles, the insertion process may cause anxiety, anxiousness, or even dissuade them from wanting to use the sensor. The embodiments described below are intended to ease anxiety associated with insertion because of needles or sharps during the insertion process. For example, in one embodiment, during the insertion process the insertion tool or insertion device is designed or configured to provide a distraction to the subject from physical sensations associated with insertion such as discomfort or pinching of skin.

The various embodiments discussed below are intended to be exemplary and should not be viewed or construed as discrete individual embodiments. Rather, where possible, individual features or elements discussed in each embodiment should be considered transferable or combinable with the various other embodiments disclosed below.

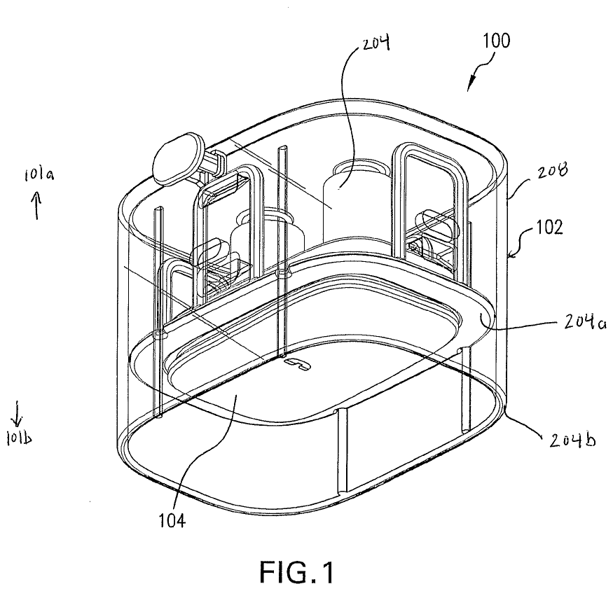

FIG. 1 is an exemplary pseudo-isometric view of select components of an insertion assembly 100, in accordance with embodiments of the present invention. In FIG. 1, the insertion assembly 100 is shown prior in a second position that is prior to inserting the sensor into a subject. The insertion assembly 100 includes an insertion device 102 that is configured to insert an implantable sensor into a subject. In preferred embodiments a portion of the implantable sensor is coupled within an on-body assembly 104. Additionally, another portion of the implantable sensor is inserted into subcutaneous tissue of the subject. The implantable sensor is typically configured to detect or measure concentration of at least one analyte within the tissue of the subject. Exemplary, non-limiting analytes include, but are not limited to glucose, lactate, tissue oxygen, ketones, and the like.

The on-body assembly includes an electronics module that provides power to the implantable sensor and also houses electronic components that interface with the implantable sensor to enable detection, recording, and data transmission from the implantable sensor coupled to the electronics module to other devices such as mobile devices or remote displays.

The arrow 101a indicates the direction toward a proximal end of the insertion assembly 100. For simplicity, the proximal end may be referenced as proximal end 101a. Similarly, the arrow 101b indicates the direction toward the distal end of the insertion assembly 100. For simplicity, the distal end may be referenced as distal end 101b. Throughout this disclosure, the proximal end 101a and the distal end 101b should be construed with respect to the arrows 101a and 101b in FIG. 1.

The insertion device 102 is an assembly that includes an encasement 208 and a slider 204. The encasement 208 includes an encasement bottom 208b and the slider 204 includes a slider bottom face 204a. Additionally, the on-body assembly 104 is illustrated as being removably coupled to the slider 204. In the second position illustrated In FIG. 1, the slider 204 and the on-body assembly are fully positioned or located within the encasement 208. Specifically, the slider bottom face 204 is located within the encasement 208.

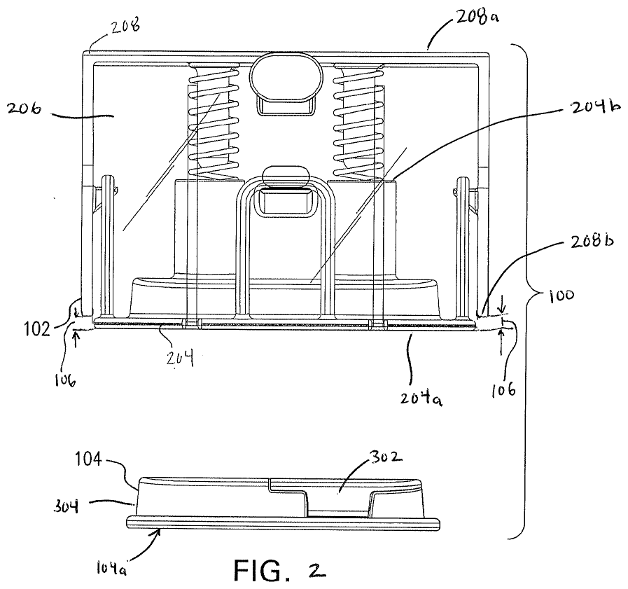

FIG. 2 is an exemplary illustration of the insertion assembly 100 in a third position, in accordance with embodiments of the present invention. In FIG. 2, the insertion assembly includes the insertion device 102 and the on-body assembly 104. The insertion assembly 100 is shown in a third position that corresponds with after the sensor has been inserted and the on-body assembly 104 has been coupled to the subject. As illustrated in FIG. 2, the insertion device 102 includes an encasement 208 and a slider 204. The encasement 208 is partially defined by an encasement top 208a and an encasement bottom 208b. The encasement top 208a and the encasement bottom 208b define an interior 206. The slider 204 includes a slider bottom face 204a located toward the proximal end 101b. The slider 204 further includes a slider top 204b. In preferred embodiments, the slider 204 is at least partially located within the interior 206. Note that in the third position, as illustrated in FIG. 2, the slider bottom face 204a extends beyond the encasement bottom 208b by a gap 106.

The on-body assembly 104 includes an electronics assembly 304 and a sensor assembly 302. The electronics assembly 304 is further defined by an on-body base 104a. As illustrated in FIG. 2, the sensor assembly 302 is coupled to the electronic assembly 304 to create the on-body assembly 104. FIG. 2 is an illustration of where the insertion process is complete, or the insertion process has been executed and note the gap 106 between an encasement 208 and slider 204. In other words, the insertion process results in a portion of the slider 204, specifically the slider bottom face 204a, protruding beyond the encasement bottom 208b of the encasement 208.

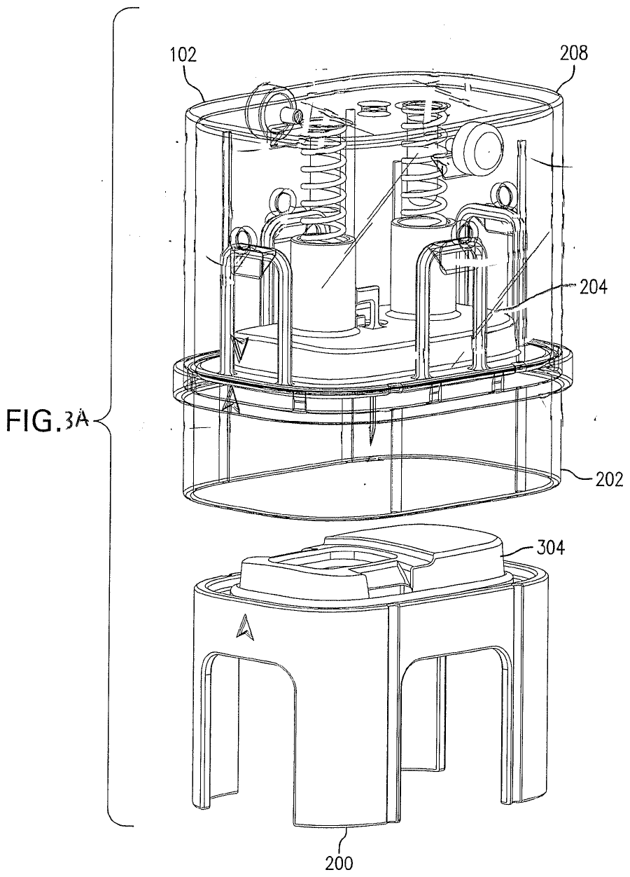

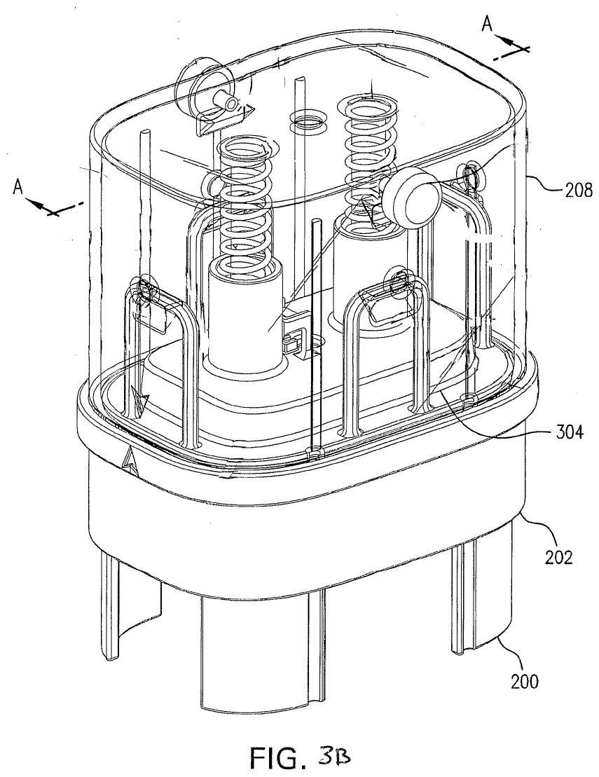

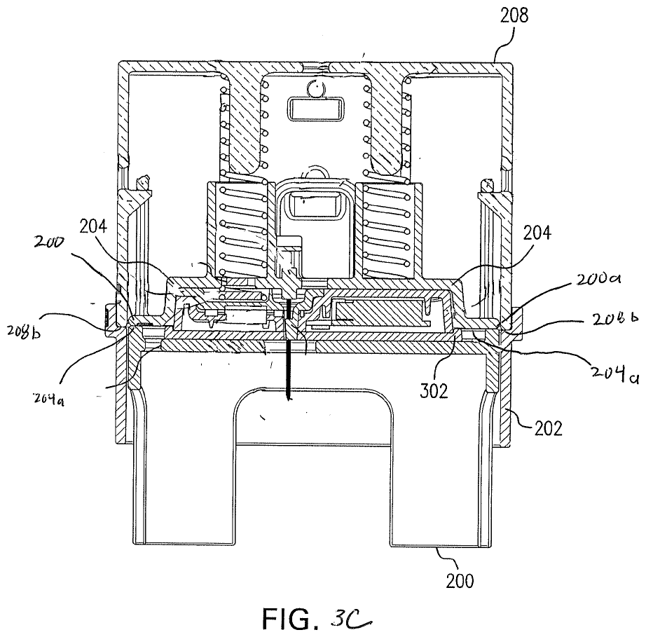

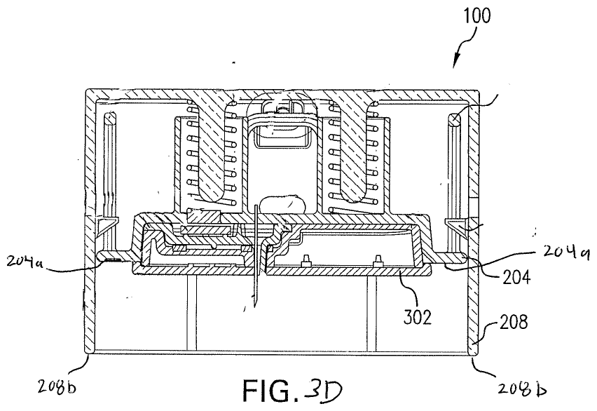

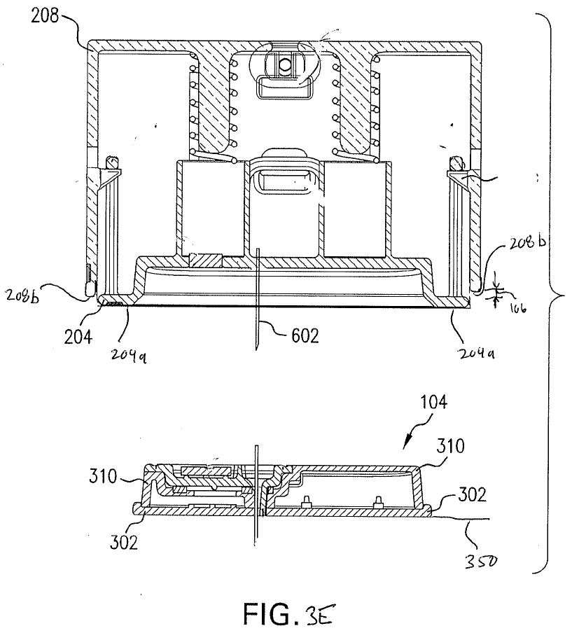

FIG. 3A is an exemplary illustration of the multiple components associated with the insertion assembly 100 during an assembly phase of an insertion process, in accordance with embodiments of the present invention. For example, there is a stand 200 that supports an electronic assembly 304. Additionally, there is a skirt 202 removably coupled to the insertion device 102. The insertion device 102 includes the encasement 208 and the slider 204 is located within the encasement 208. Not visible in FIG. 3A is the sensor assembly (302) that is removably coupled to the slider 204. During the assembly phase of insertion the slider 204 may be in a first position. In many embodiments, when in the first position the slider bottom face 204a is substantially even or coincident with the bottom of the encasement bottom 208b.

The skirt 202 is intended to minimize exposure of the sharp 602. Additionally, the skirt 202 is sized to encompass, cover, or envelop the stand 200. Positioned on the stand 200 is the electronic assembly 304. As illustrated in FIG. 3A, the on-body assembly in FIG. 2 is separated into discrete components of the sensor assembly 302 (removably coupled within the slider 204), and the electronic assembly 304.

FIGS. 3B and 3C are exemplary illustrations of multiple components associated with the insertion assembly 100 during a coupling phase, in accordance with embodiments of the present invention. FIG. 3B is a pseudo-isometric view with the encasement 208 illustrated as being transparent. FIG. 3C a cross-section view A-A from FIG. 3B. The coupling phase of insertion illustrated in FIGS. 3B and 3C is intended to couple the sensor assembly 306 to the electronics assembly 304. During the coupling phase the slider 204 remains substantially level or even with the bottom of the encasement 208 and the sensor assembly 306 is secured or attached to the electronics assembly 304.

In both FIGS. 3B and 3C the encasement 208 and skirt 202 have been removably coupled with the stand 200. As illustrated, the encasement 208 includes the slider 204 which in turn includes the sensor assembly 306. As illustrated in FIG. 3A, the electronics assembly 304 is positioned or located on the stand 200. Accordingly, coupling the encasement 208 and skirt 202 with the stand 200 aligns the sensor assembly 306 with the electronics assembly 304. In many embodiments the removable coupling of the encasement 208 and skirt 202 with the stand 200 is accomplished via a friction fit.

The coupling of the encasement 208 with the stand also locates the sharp 602 and sensor through the electronics assembly 304. As illustrated in FIGS. 3B and 3C, the slide 204 may be considered in the first position where the slider bottom face 204a is substantially even or coincident with the encasement bottom 208b. The first position is illustrated in FIG. 3C where the slider bottom face 204a is illustrated as being coincident with both the encasement bottom 208b and a top surface 200a of the stand 200.

FIG. 3D is an exemplary illustration of select components of the insertion assembly 100 in a second position, in accordance with embodiments of the present invention. In FIG. 3D the slider 204 within the insertion assembly 100 has been transitioned to the second position. In many embodiments the action to transition the insertion assembly 100 from the first position to the second position is depressing the encasement 208 in a distal direction to load energy storage 206a. Additionally, compressing the encasement 208 over the stand 200 removably couples the electronics assembly 304 to the sensor assembly 302. In preferred embodiments, plastic snaps are used to removably couple the sensor assembly 302 and the electronics assembly together. In the second position illustrated in FIG. 3D, the slider 204 is entirely disposed within the encasement 208. Specifically, the slider bottom face 204a has been displaced in a proximal direction within the encasement and away from the encasement bottom 208b.

In the second position the insertion assembly 100 may be considered “loaded”. For example, upon being placed in the second position, the insertion assembly 100 includes an on-body assembly 104 that is removably coupled to the slider 204. Moreover, in the second position the on-body assembly 104 that was previously two separate assemblies (the sensor assembly 302 and the electronics assembly 304) are coupled together. Additionally, the energy storage 206a has been compressed resulting in the slider 204 being ready to translate to the third position.

FIG. 3E is an exemplary illustration of select components of the insertion assembly 100 in a third position, in accordance with embodiments of the present invention. FIG. 3E is intended to illustrate the insertion assembly 100 after the insertion process has been completed. In FIG. 3E, the on-body assembly 104 is removably coupled to a subject and the insertion device 102. In some embodiments, an adhesive patch is coupled to the on-body assembly 104 and the adhesive patch secures the on-body assembly 104 to the subject. As illustrated in FIG. 3E, the insertion device 102 has been lifted in a proximal direction away from the on-body assembly 104. Separation of the on-body assembly 104 from the encasement 208 and slider 204 illustrates that in the third position, the slider bottom face 204a is extended beyond the encasement bottom 208b by the gap 106.

The transition from the second position to the third position results in projecting the slider bottom face 204a beyond the encasement bottom 208. The intent of extending the slider bottom face 204a beyond the first position is to provide a best opportunity for the adhesive patch of the on-body assembly to bond with the subject. Additionally, an additional benefit of allowing or permitting the slider bottom face 204a to contact the subject is a physical distraction from the sensation of the sharp piercing the skin of the subject during insertion of the sensor.

The embodiments described above should not be construed as limiting. The sensor array should not be perceived as limited to subcutaneous placement for measurement of glucose, tissue oxygen and lactate. Other embodiments, for use in diagnosing or determining risk score for other conditions or diseases can employ various sensors to measure other combinations of analytes in different locations within the subject.

In many embodiments, additional features or elements can be included, added or substituted for some or all of the exemplary features described above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. An insertion assembly to implant a sensor, comprising:
an insertion device that includes an encasement having an interior defined between an encasement top and an encasement bottom;
a slider having a slider top and a slider bottom face, the slider top being located within the interior of encasement, the slider initially being in a first position, transitioning to a second position and ending in a third position,
wherein in the first position the slide bottom face is coincident with the encasement bottom, in the second position the slide bottom face is fully contained in the interior, and in the third position, the slide bottom face extends beyond the encasement bottom by a gap.

2. The insertion assembly according claim 1, wherein a sharp is mounted to the slider.

3. The insertion assembly according to claim 2, further comprising:
a sensor assembly being detachably coupled to the slider in the first position and the second position.

4. The insertion assembly according to claim 3, wherein in the second position, an electronic assembly is coupled to the sensor assembly to form an on-body assembly.

5. The insertion assembly according to claim 4, wherein the on-body assembly is detachably coupled to the slider.

6. The insertion assembly according to claim 5, wherein in the second position, the sharp extends through the sensor assembly and the electronic assembly.

7. The insertion assembly according to claim 4, wherein in the third position, the on-body assembly is decoupled from the slider.

8. The insertion assembly according to claim 1, wherein in the first position the slider top is positioned within the encasement bottom.

9. The insertion assembly according to claim 8, wherein in the second position, the slider top remains positioned within the encasement bottom.

10. The insertion assembly according to claim 9, wherein in the third position, the slider top remains positioned within the encasement bottom.
